(19) United States
(12) Patent Application Publication
(10) Pub. No.: US 2026/0306871 A1
(43) Pub. Date: Oct. 8, 2026

(54) JAUNDICE TREATING SWADDLE

(71) Applicant: Zechariah Horton, Thayer, MO (US)

(72) Inventor: Zechariah Horton, Thayer, MO (US)

(21) Appl. No.: 19/171,917

(22) Filed: Apr. 7, 2025

(51) Int. Cl. A61N 5/06

(52) U.S. Cl. CPC A61N 5/0621; A61N 2005/0636; A61N 2005/0645; A61N 2005/0662

(57) ABSTRACT

A jaundice treating swaddle to wrap around an infant, the jaundice treating swaddle including a main body within which the infant is wrapped when the main body is in a wrapped configuration, a power cable connected to the main body to provide electrical power from an electrical outlet to the jaundice treating swaddle, and a plurality of fluorescent lights disposed on a first surface of the main body to illuminate in response the power cable receiving electrical power from the electrical outlet.

### BACKGROUND

### 1. Field

The present general inventive concept relates generally to a swaddle, and particularly, to a jaundice treating swaddle.

### 2. Description Of The Related Art

Jaundice is a common condition in infants, often requiring phototherapy to break down elevated bilirubin levels.

However, conventional treatment methods for jaundice in infants can be distressing for both the baby and the caregivers, as they often involve exposing the baby to harsh lights in a clinical setting, which can feel impersonal and uncomfortable.

Therefore, there is a need for a device and/or process of treating jaundice by integrating continuous phototherapy within a soft, adjustable swaddle, allowing for a more nurturing environment and experience for an infant.

### SUMMARY

The present general inventive concept provides a jaundice treating swaddle.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a jaundice treating swaddle to wrap around an infant, the jaundice treating swaddle including a main body within which the infant is wrapped when the main body is in a wrapped configuration, a power cable connected to the main body to provide electrical power from an electrical outlet to the jaundice treating swaddle, and a plurality of fluorescent lights disposed on a first surface of the main body to illuminate in response the power cable receiving electrical power from the electrical outlet.

The jaundice treating swaddle may further include a power distributing cable to receive the electrical power from the power cable and to distribute the electrical power to the plurality of fluorescent lights to enable the plurality of fluorescent lights toilluminate.

The jaundice treating swaddle may further include a first set of hook and loop connectors disposed at a first side and on the first surface of the main body, and a second set of hook and loop connectors disposed at a second side and on a second surface of the main body to correspond to and connect to the first set of hook and loop connectors when the main body is in the wrapped configuration.

The infant wrapped within the main body may receive jaundice-treating benefits from the fluorescent lights when the fluorescent lights are illuminated.

The main body may be constructed from a flexible material that is also soft and warm, and which further includes bamboo.

### BRIEF DESCRIPTION OF THE DRAWINGS

These and/or other features and utilities of the present generally inventive concept will become apparent and more readily appreciated from the following description of the embodiments, taken in conjunction with the accompanying drawings ofwhich:

FIG. 1 illustrates a view of a jaundice treating swaddle in an unwrapped configuration, according to an exemplary embodiment of the present general inventive concept; and

FIG. 2 illustrates a view of the jaundice treating swaddle of FIG. 1 in a wrapped configuration, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Jaundice Treating Swaddle 100
Main Body 110
First Side 111
First Surface 112
Second Side 113
Second Surface 114
First Set of Hook and Loop Connectors 120
Second Set of Hook and Loop Connectors 130
Fluorescent Lights 140
Power Cable 150
Plug 151
Power Distributing Cable 160
Adjustable Strap 170

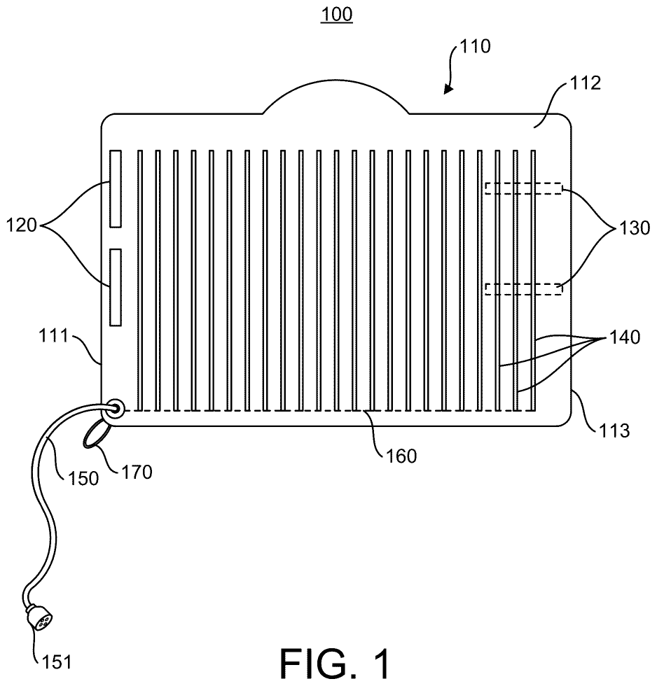

FIG. 1 illustrates a view of a jaundice treating swaddle 100 in an unwrapped configuration, according to an exemplary embodiment of the present general inventiveconcept.

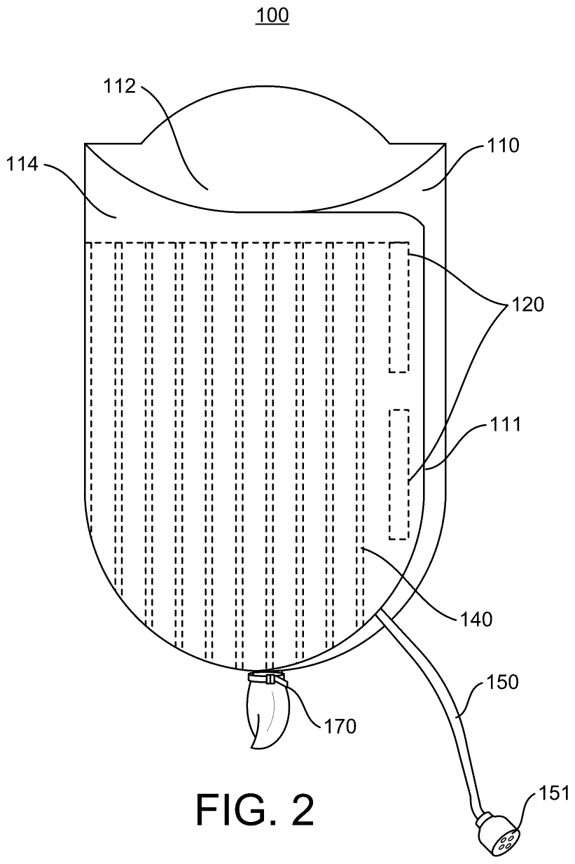

FIG. 2 illustrates a view of the jaundice treating swaddle 100 of FIG. 1 in a wrapped configuration, according to an exemplary embodiment of the present general inventive concept.

The designer hamper assembly 100, and all components therein, may be constructed from at least one of wood, bamboo, plastic, paper, metal, silicone, liquid, fibers, threads, cloth, fabric, foam, sponge, nylon, vinyl, feathers, glass, adhesives, circuitry, and rubber, etc., but is not limited thereto, and can be constructed from any material known to one of ordinary skill in the art.

The jaundice treating swaddle 100 may include a main body 110, a first set of hook and loop connectors 120, a second set of hook and loop connectors 130, a plurality of fluorescent lights 140, a power cable 150, a power distributing cable 160, and an adjustable strap 170, but is not limited thereto.

Referring to FIGS. 1 and 2, the jaundice treating swaddle 100 may include the main body 110 to be flexible, soft, and comfortable, so that the main body 110 can easily and comfortable wrap around an infant.

The first set of hook and loop connectors 120 may be disposed at a first side 111 of the main body 110, and may be disposed on a first surface 112 of the main body 110.

The second set of hook and loop connectors 130 may be disposed at a second side 113 of the main body 110 that is opposite from the first side 111 of the main body 110, and may be disposed on a second surface 114 of the main body 110 opposite from the first surface 112 of the main body 110.

The first set of hook and loop connectors 120 may be configured as vertical strips disposed on the first surface 112 of the main body 110, and may correspond and connect to the second set of hook and loop connectors 130 that may be configured as horizontal strips disposed on the second surface 114 of the main body 110, such that a space created within the jaundice treating swaddle 100 when the main body 100 is in the wrapped configuration, as illustrated in FIG. 2, may be adjustable in size. Thus, infants of various sizes may be wrapped within the jaundice treating swaddle 100.

The first surface 112 may be an inner surface of the main body 110, and the second surface 114 may be an outer surface of the main body 110.

The plurality of fluorescent lights 140 may be disposed to be parallel to each other on the first surface 112 of the main body 110, so that when the main body 110 is in the wrapped configuration as illustrated in FIG. 2, an infant wrapped within the main body 110 may receive jaundice-treating benefits of the fluorescent lights 140 when the fluorescent lights 140 are illuminated.

The power cable 150 may be configured to provide electric power to the plurality of fluorescent lights 140, and may include a plug 151 at a first end of the power cable 150 that may be plugged into any type of electrical socket known to one of ordinary skill in the art.

The power cable 150 may be connected at a second end to the power distributing cable 160, which may distribute power provided from the electrical socket through the plug 151 via the power cable 150 to each of the plurality of fluorescent lights 140.

Thus, when the plug 151 is plugged into an electrical socket, power may flow through the plug 151, through the power cable 150, then through the power distributing cable 160, which distributes the power to each of the plurality of fluorescent lights 140, thus causing each of the plurality of fluorescent lights 140 to illuminate.

The adjustable strap 170 may be disposed at a bottom portion of the main body 110 to allow the main body 110 to remain tied up in the wrapped configuration, as illustrated in FIG. 2.

The present general inventive concept may include a jaundice treating swaddle 100 to wrap around an infant, the jaundice treating swaddle 100 including a main body 110 within which the infant is wrapped when the main body 110 is in a wrapped configuration, a power cable 150 connected to the main body 110 to provide electrical power from an electrical outlet to the jaundice treating swaddle 100, and a plurality of fluorescent lights 140 disposed on a first surface 112 of the main body 110 to illuminate in response the power cable 150 receiving electrical power from the electricaloutlet.

The jaundice treating swaddle may further include a power distributing cable 160 to receive the electrical power from the power cable 150 and to distribute the electrical power to the plurality of fluorescent lights 140 to enable the plurality of fluorescent 140 lights to illuminate.

The jaundice treating swaddle 100 may further include a first set of hook and loop connectors 110 disposed at a first side 111 and on the first surface 112 of the main body 110, and a second set of hook and loop connectors 130 disposed at a second side 113 and on a second surface 114 of the main body 110 to correspond to and connect to the first set of hook and loop connectors 120 when the main body 110 is in the wrappedconfiguration.

The infant wrapped within the main body 110 may receive jaundice-treating benefits from the fluorescent lights 140 when the fluorescent lights 140 are illuminated.

The main body 110 may be constructed from a flexible material that is also soft and warm, and which further includes bamboo.

A primary purpose of the present general inventive concept serves is to provide effective treatment for jaundice in newborns while prioritizing their comfort and security. The present general inventive concept reimagines the process of treating jaundice by integrating continuous phototherapy within a soft, adjustable swaddle, allowing for a more nurturing environment. This innovative approach not only addresses the medical needs of the infant but also enhances the emotional experience for families, fostering a sense of closeness and care during a challenging time. The present general inventive concept includes several thoughtfully designed components that enhance its functionality and user experience. The bamboo swaddle is crafted from a thin, breathable fabric, ensuring that the baby remains comfortable and secure while also allowing for easy movement and facilitating feeding. This is particularly important for maintaining the baby's well-being during treatment, as it promotes a sense of safety. The Velcro fastenings provide adjustable options for a snug fit, minimizing fussiness and discomfort, which can often arise during medical treatments. The embedded blue light features adjustable fluorescent strips strategically placed inside the swaddle. This design allows for targeted phototherapy that effectively breaks down bilirubin levels without exposing the infant to harsh external lighting, creating a calming atmosphere. Additionally, the fiber-optic cable supports the lighting system, ensuring consistent and reliable treatment throughout the duration of phototherapy. A tie system further customizes the fit, enhancing both safety and comfort for the baby, which is crucial during medical interventions. The inclusion of a USB charging port is another key feature, allowing for convenient recharging of the lights. This enhances the portability of the present general inventive concept, enabling parents to use it in various settings, whether at home or while on the go. The present general inventive concept effectively tackles several critical issues associated with traditional jaundice treatment methods. One of the main concerns for caregivers is the comfort and security of the infant during treatment. Traditional phototherapy devices often leave babies uncovered and stationary, which can lead to distress and discomfort. In contrast, the present general inventive concept allows infants to be securely swaddled, promoting a sense of safety and comfort that is vital during this medical process. Moreover, the design enables parents to hold, feed, and comfort their baby without interruption, significantly reducing stress for both the baby and the caregivers. The ability to maintain physical contact is crucial for the emotional well-being of both the infant and the parent. Considering traditional blue lighting in hospitals can cause discomfort for both infants and their parents; the present general inventive concept's embedded lighting minimizes exposure to harsh external lights, creating a more soothing healing environment.

Most importantly, the integrated blue light effectively treats jaundice by breaking down bilirubin levels while ensuring that the baby receives necessary medical care in a nurturing manner. By combining effective medical treatment with a design that prioritizes comfort and emotional support, the present general inventive concept significantly improves the overall treatment experience, addressing both the physical and emotional challenges faced by families during this critical time.

The present general inventive concept stands out for its innovative combination of effective jaundice treatment and a comforting swaddle design, allowing for continuous phototherapy in a secure and nurturing environment. Unlike traditional phototherapy devices that often leave babies exposed and stationary, the present general inventive concept features adjustable blue fluorescent lighting embedded within a soft bamboo swaddle, ensuring targeted treatment while keeping the infant snug and mobile. This design not only promotes better healing by breaking down bilirubin levels but also enhances the overall experience for both babies and parents, making it easier to hold and comfort the child during a crucial time.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A jaundice treating swaddle to wrap around an infant, the jaundice treating swaddle comprising:
a main body within which the infant is wrapped when the main body is in a wrapped configuration;
a power cable connected to the main body to provide electrical power from an electrical outlet to the jaundice treating swaddle; and
a plurality of fluorescent lights disposed on a first surface of the main body to illuminate in response the power cable receiving electrical power from the electricaloutlet.

2. The jaundice treating swaddle of claim 1, further comprising:
a power distributing cable to receive the electrical power from the power cable and to distribute the electrical power to the plurality of fluorescent lights to enable the plurality of fluorescent lights to illuminate.

3. The jaundice treating swaddle of claim 2, further comprising:
a first set of hook and loop connectors disposed at a first side and on the first surface of the main body; and
a second set of hook and loop connectors disposed at a second side and on a second surface of the main body to correspond to and connect to the first set of hook and loop connectors when the main body is in the wrapped configuration.

4. The jaundice treating swaddle of claim 2, wherein the infant wrapped within the main body receives jaundice-treating benefits from the fluorescent lights when the fluorescent lights are illuminated.

5. The jaundice treating swaddle of claim 1,wherein the main body is constructed from a flexible material that is also soft and warm, and which further comprises bamboo.
